(19) United States
(12) Patent Application Publication
(10) Pub. No.: US 2002/0123077 A1
(43) Pub. Date: Sep. 5, 2002

(54) Novel compounds capable of modulating biofilms

(76) Inventors: George A. O'Toole, Hanover, NH (US); Roberto Kolter, Chestnut Hill, MA (US); Mary Ellen Davey, Post Mills, VT (US); David H. Blank, Hanover, NH (US); Nicky C. Caiazza, Lebanon, NH (US); Deborah A. Hogan, Boston, MA (US)

Correspondence Address: CLARK & ELBING LLP, 101 FEDERAL STREET, BOSTON, MA, 02110, US

(21) Appl. No.: 09/966,826

(22) Filed: Sep. 28, 2001

Related U.S. Application Data
(60) Provisional application No. 60/236,639, filed on Sep. 29, 2000.

(51) Int. Cl. G01N 33/53; G01N 33/567; C07K 1/00; C07K 14/00; C07K 17/00

(52) U.S. Cl. 435/7.2; 530/395

(57) ABSTRACT

The invention features a novel method for identifying compounds capable of affecting a microbial biological activity, such as biofilm formation, development and dissolution. The method includes: (a) obtaining supernatant from a closed culture system that contains at least one type of microorganism; (b) exposing a target organism to the supernatant or an extract thereof; and (c) measuring the level of the biological activity. Compounds identified by this method are provided, as wells as methods for disrupting biofilms and inhibiting or promoting their formation.

This application claims priority from U.S. Ser. No. 60/236,639, which was filed on Sep. 29, 2000.

### STATEMENT AS TO FEDERALLY SPONSORED RESEARCH

This invention was made with Government support under grant Nos. GM58213 and AI07519 awarded by the NIH. The Government has certain rights in the invention.

### FIELD OF THE INVENTION

The present invention relates to methods for identifying compounds that are capable of modulating microbial biological activity, such as the formation, development and dissolution of microbial biofilms, and related compounds

### BACKGROUND OF THE INVENTION

Biofilms are complex communities of surface-attached microorganisms, comprised either of a single or multiple species. Over the past few decades, there has been a growing realization that bacteria in most environments are found predominantly in biofilms, not as planktonic cells such as those typically studied in the laboratory. This realization has spurred much research into the physical and chemical properties of biofilms, their morphology, and the mechanism of their development.

Biofilms can form in almost any hydrated environment that has the proper nutrient conditions, and can develop on a wide variety of abiotic (both hydrophobic and hydrophilic) and biotic (e.g., eukaryotic cells) surfaces. The formation of biofilms is an important aspect of normal development for many microbial species. Biofilm development begins when a group of individual cells make the transition from planktonic (free-swimming) existence to a lifestyle in which the microorganisms are firmly adhered to a surface. After their initial attachment to the surface, the cells are believed to undergo a series of physiological changes resulting in a highly structured, sessile multi-cellular community. The developmental cycle is completed when planktonic cells are shed from the biofilm into the medium, perhaps in response to a lack of sufficient nutrients, or microbially-produced factors. This cycle, shown in FIG. 1, is believed to be a highly regulated process under the control of a complex signal transduction regulatory circuitry that senses and responds to environmental cues and is modulated by extracellular factors.

The formation of biofilms can have serious negative consequences in medical, industrial, and natural settings, resulting in high costs both in human health and economic terms. Biofilm-associated infections extend hospital stays an average of about three days and cost in excess of a billion dollars per year (Archibald, et al.,1997, Nosocomial Inf. 11(2):245-255; Bryers, 1991, TIBTECH 9:422-426; Costerton, 1995, J. Indus. Microbiol. 15:137-140). In clinical settings, biofilms can form on a variety of surfaces. For example, Pseudomonas aeruginosa, an organism that causes nosocomial infections, forms biofilms on surfaces as diverse as cystic fibrosis lung tissue, contact lenses, and catheter lines. Biofilms formed on indwelling medical devices serve as a reservoir of bacteria that can be shed into the body, leading to a chronic systemic infection. Indeed, up to 82% of nosocomial bacteremias are the result of bacterial contamination of intravascular catheterizations (Archibald, Supra).

Furthermore, biofilm bacteria are much more resistant to treatment with antimicrobial compounds than planktonic bacteria, making them more resistant to treatment with antibiotics and biocides. In some cases, biofilm-grown bacteria can become up to 1000-fold more resistant to an antibiotic than their planktonic counterparts (Hoyle, B. D., et al., 1991, Progress in Drug Research. 37:91-105). Thus there is a need to develop methods for identifying compounds that are able to kill bacteria in the biofilm form, as well as compounds capable of altering or disrupting biofilms and biofilm development in order to eradicate biofilms in both clinical and industrial settings, to render bacteria more susceptible to conventional anti-microbial treatments or natural immune response, and to promote biofilms in agricultural, industrial bioprocessing or environmental settings.

### SUMMARY OF THE INVENTION

In a first aspect, the present invention features a method for identifying a compound capable of affecting a microbial biological activity, such as biofilm formation or attachment on a surface. The method involves a) obtaining supernatant from a closed culture system, b) exposing a target organism to the supernatant; and c) measuring the level of the biological activity of interest. The method can be used to identify compounds that inhibit or promote the formation of biofilms, or compounds that disrupt preexisting biofilms. Compounds can be identified which inhibit biofilm formation at various stages, including initiation (attachment to a surface) and development. The method can also be used to identify compounds that are capable of killing microorganisms within a biofilm or to identify compounds that potentiate the activity of other antibiotics to kill microorganisms within a biofilm.

In various embodiments of the first aspect of the invention, the exposing step occurs before, after, or at the same time as inoculation of a culture medium with the target organism. When testing for compounds that are capable of preventing or promoting biofilm formation, it is preferable to expose the target microorganism to the supernatant prior to formation of a biofilm. When testing for compounds that disrupt preexisting biofilms, the exposing step preferably occurs after the target microorganism has formed a biofilm.

In another aspect, the invention features methods for preventing or promoting the formation of a biofilm or disrupting a preexisting biofilm. These methods involve exposing the target organism to supernatant from a closed culture system or to an extract thereof, or to purified compounds derived therefrom, or to chemically synthesized compounds. This process can also be used as a method of potentiating or otherwise modulating a biofilm-associated activity.

The microorganism from which the supernatant is obtained (i.e., the “source microorganism”) may either be the same or different as the target organism and may be an archaeal, bacterial, fungal, protozoan, or algal species. In a preferred embodiment of the invention, the microorganism of the closed culture system and/or the target organism is a bacterial organism selected from the group consisting of: Pseudomonas fluorescens, Pseudomonas aeruginosa, Pseudomonas acidovorans, Pseudomonas alcaligenes, Pseudomonas putida, Pseudomonas syringae, Pseudomonas aureofaciens, Pseudomonas fragi, Fusobacterium nucleatum, Treponema denticola, Porphyromonas gingivalis, Moraxella catarrhalis, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudo tuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Pasteurella multocida, Pasteurella haemolytica, Helicobacter pylori, Campylobacter fetus, Campylobacter jejuni, Campylobacter coli, Borrelia burgdorferi, Vibrio cholerae, Vibrio paramaemolyticus, Legionella pneumophila, Listeria monocytogenes, Neisseria gonorrhoeae, Neisseria meningitidis, Gardnerella vaginalis, Bacteroides spp., Clostridium difficile, Mycobacterium tuberculosis, Mycobacterium avium, Mycobacterium intracellulare, Mycrobacterium leprae, Corynebacterium diphtheriae, Corynebacterium ulcerans, Streptococcus spp., Enterococcus spp., Desulfvibrio spp., Actinomyces spp., Erwinia spp., Xanthomonas spp., Xylella spp., Clavibacter spp., Desulfomonas spp., Desulfovibrio spp., Desulfococcus spp., Desulfobacter spp., Desulfobulbus spp., Desulfosarcina spp., Deslfuromonas spp., Bacillus spp., Streptomyces spp., Clostridium spp., Rhodococcus spp., Thermatoga spp., Sphingomonas spp., Zymomonas spp., Micrococcus spp., Azotobacter spp., Norcardia spp., Brevibacterium spp., Alcaligenes spp., Microbispora spp., Micromonospora spp., Methylobacterium organophilum, Pseudomonas reptilivora, Pseudomonas carragienovora, Pseudomonas dentificans, Corynebacterium spp., Propionibacterium spp., Xanothomonas spp., Methylobacterium spp., Chromobacterium spp., Saccharopolyspora spp., Actinobacillus spp., Alteromonas spp., Aeronomonas spp., Agrobacterium tumefaciens, Staphylococcus aureus, Staphylococcus epidennidis, Staphylococcus hominis, Staphylococcus haemolyticus, Staphylococcus warneri, Staphylococcus cohnii, Staphylococcus saprophyticus, Staphylococcus capitis, Staphylococcus lugdunensis, Staphylococcus intennedius, Staphylococcus hyicus, Staphylococcus saccharolyticus and Rhizobium spp., and mutant derivatives thereof. In a particularly preferred embodiment, the bacterial organism is a coagulase-negative Staphylococcus.

In another preferred embodiment, the source microorganism and/or the target organism is a fungal organism selected from the group consisting of Absidia spp., Actinomadura madurae, Actinomyces spp., Allescheria boydii, Altemaria spp., Anthopsis deltoidea, Aphanomyces spp., Apophysomyces eleqans, Armillaria spp., Arnium leoporinum, Aspergillus spp., Aureobasidium pullulans, Basidiobolus ranarum, Bipolaris spp., Blastomyces dernatitidis, Botrytis spp., Candida spp., Centrospora spp., Cephalosporium spp., Ceratocystis spp., Chaetoconidium spp., Chaetomium spp., Cladosporium spp., Coccidioides immitis, Colletotrichum spp, Conidiobolus spp., Corynebacterium tenuis, Cryptoporiopsis spp., Cylindrocladium spp., Cryptococcus spp., Cunninghamella bertholletiae, Curvularia spp., Dactylaria spp., Diplodia spp., Epidermophyton spp., Epidennophyton floccosum, Exserophilum spp., Exophiala spp., Fonsecaea spp., Fulvia spp., Fusarium spp., Geotrichum spp., Guignardia spp., Helminthosporium spp., Histoplasma spp., Lecythophora spp., Macrophomina spp., Madurella spp., Magnaporthe spp., Malassezia furfur, Microsporum spp., Monilinia spp., Mucor spp., Mycocentrospora acerina, Nectria spp., Nocardia spp., Oospora spp., Ophiobolus spp., Paecilomyces spp., Paracoccidioides brasiliensis, Penicillium spp., Phaeosclera dematioides, Phaeoannellomyces spp., Phialemonium obovatum, Phialophora spp., Phlyctaena spp., Phoma spp., Phomopsis spp., Phymatotrichum spp., Phytophthora spp., Pythium spp., Piedraia hortai, Pneumocystis carinii, Puccinia spp., Pythium insidiosum, Rhinocladiella aquaspersa, Rhizomucor pusillus, Rhizoctonia spp., Rhizopus spp., Saccharomyces spp., Saksenaea vasifornis, Sarcinomyces phaeomurifonnis, Scerotium spp., Sclerotinia spp., Sphaerotheca spp., Sporothrix schenckii, Syncephalastrum racemosum, Taeniolella boppii, Taphrina spp., Thielaviopsis spp., Torulopsosis spp., Trichophyton spp., Trichosporon spp., Ulocladium chartarum, Ustilago spp., Venturia spp., Verticillium spp., Wangiella dennatitidis, Whetxelinia spp., Xylohypha spp., and their synonyms.

The supernatant may be harvested at any stage of microbial growth. In one embodiment of the invention, the supernatant from the closed culture system is spent supernatant obtained at the stationary phase. In alternative embodiments, the supernatant is obtained at early, mid, and/or late exponential phase.

The source microorganism of the closed culture system may be grown on a surface, planktonically, or both. The closed culture system may be pure culture that includes only one strain or species of microorganism. Alternatively, the system may include a mixture of species.

In another preferred embodiment, the method of the invention includes two or more closed culture systems. Each of these systems preferably contains a different strain of source microorganism or a different set of environmental conditions. The target organism is then exposed to the supernatant of each of the individual culture systems, separately. The target organism may be comprised of a single species or multiple species of microbial organisms.

The present invention also features a novel biological surfactant that is capable preventing the formation of biofilms and disrupting preexisting biofilms. This surfactant is a rhamnolipid compound that includes a chain of at least three rhamnose moieties linked to a lipid moiety and derivatives thereof. In a preferred embodiment, the chain has seven rhamnose moieties and the lipid moiety is a complex lipid containing an ester linkage.

The compound of the invention is preferably obtained from the supernatant of a closed culture system comprising P. aeruginosa, but may also be synthesized using known techniques. The invention also provides biofilm-modulating compounds identified by the method described above, wherein the compound is not an acyl homoserine lactone, a microbially-produced hydrolytic enzyme, a Lactobocillus biosurfactant, or an autoinducing compound.

Definitions

By “biofilm” is meant a population of microorganisms comprised of a single species or multiple species that are adhered to an abiotic or biotic surface, or to each other, or at any interface.

By “biological activity” is meant an activity associated with a microbial organism, including the formation, development, and dissolution of biofilms, or a property or phenotype associated with a biofilm.

By “closed culture system” or “closed system” is meant a culture system in which growth of a microorganism occurs in a chamber containing culture medium in which the accumulation of microbially-produced factors is allowed to occur. A closed culture system may be produced by inoculating a closed culture vessel containing a single batch of medium with at least one species of microorganism. This includes growing microbes in batch culture (including a fed batch culture) in a microtiter dish, test tube, flask, or fermenter, either with or without agitation. The cells of the closed culture system may be grown to various stages, including lag, early-exponential, mid-exponential, late-exponential, early stationary, and late stationary phases. The closed culture system may be either an aerobic or anaerobic environment and may include any of a wide variety of media depending upon the microorganisms being grown.

The closed culture system of the invention may include, but is not limited to, any of the following: a single species of a known microorganism; a single species of an unknown organism; a mixture of two or more known organisms; a mixture of two or more unknown organisms; a mixture of at least one known organism with one or more unknown organisms; raw environmental samples from pristine environments (e.g., from soil, aquatic, rhizosphere; rhizoplane); raw environmental samples from human-impacted environments (toxic sites, industrial sites, agriculture, waste water treatment plants, etc.); and environmental samples enriched for particular groups of organisms.

By “expose” is meant to allow contact between a substance, including a compound, culture supernatant, or extract thereof, and a microorganism or target organism.

By “environment” is meant the habitat or living conditions of a population of microorganisms, such as source microorganisms or target organisms.

By “extract” is meant a product obtained from treating supernatant of a closed culture system to at least one purification step of any kind. In a preferred embodiment, the purification is designed to isolate or increase the concentration of a biofilm modulating compound or remove undersirable elements within the supernatant.

By “microorganism,” “microbial organism,” or “microbe” is meant a microscopic, single-celled organism that may either live independently or as part of a multi-cellular community or colony. The major groups of microorganisms include archaea, bacteria, fungi, protozoa, and algae.

By “modulating” is meant changing, by increase, decrease or otherwise. The change may be in amount, timing, or any other parameter.

By “supernatant” is meant media in which a microorganism has grown for some period of time. This includes the liquid portion of a culture system that is preferably substantially free of microorganisms. In a preferred embodiment, the supernatant is harvested by spinning the culture in a centrifuge to obtain a pellet of intact cells with a liquid layer (i.e., the supernatant) lying above the pellet, followed by filtering the liquid layer to remove any remaining unpelleted microbial cells. Other methods that separate the fluid portion of the culture system from the cells may alternatively be used.

By “spent supernatant” is meant supernatant that is obtained from a microorganism at or near the stationary phase of growth.

By “continuous culture system” or “continuous system” is meant a culture system with constant environmental conditions maintained through continual or continuous provision of fresh nutrients and removal of waste materials.

By “stationary phase” is meant the phase of microbial growth in a closed culture system when population growth ceases and total viable cell count plateaus or drops.

By “exponential phase” is meant the phase of microbial growth during which the microbial population is growing at a constant and maximum rate, dividing and doubling at regular intervals (i.e., log phase growth).

By “source microorganism” is meant a microorganism grown in a closed culture system from which supernatant is harvested. By “target organism” is meant either (1) a microorganism that is surveyed for the effect of a supernatant or extract thereof on its biological activities, or (2) a microorganism the biological activity of which is desired to be altered. The source or target organism may include, but is not limited to, any of the following: a single species of a known microorganism; a single species of an unknown organism; a mixture of two or more known organisms; a mixture of two or more unknown organisms; a mixture of a least one known organism with one or more unknown organisms; raw environmental samples from pristine environments (e.g., from soil, aquatic, rhizosphere; rhizoplane); raw environmental samples from human-impacted environments (toxic sites, industrial sites, agriculture, waste water treatment plants, etc.); and environmental samples enriched for particular groups of organisms.

### BRIEF DESCRIPTION OF THE DRAWINGS

FIG. 1 is a model for biofilm development. In the initial phase of biofilm formation, individual planktonic cells attach to surfaces in response to environmental cues. Establishment of a monolayer of cells is followed by formation of microcolonies and subsequently the development of a multi-layered, mature biofilm. Planktonic cells are shed from the mature biofilm to complete the cycle.

FIG. 2 is a graph showing biofilm formation as assayed in a 96 well dish over a time period of 48 hrs. The extent of biofilm formation over time first increases and then decreases.

FIG. 3 is a photograph showing a biofilm formed at 8 and 48 hrs by P. aeruginosa in the wells of a PVC microtiter dish.

FIG. 4A is a plot showing the biofilm formed after addition of spent supernatant or M63 medium (control).

FIG. 4B is a series of phase-contrast micrographs of a pre-formed biofilm treated with M63 or spent supernatant. The spent supernatant may contain one or more activities that contribute to the observed effect.

FIG. 5A is the proposed structure of a glycolipid surfactant isolated from spent culture supernatant of P. aeruginosa.

FIG. 5B is a photograph showing a water drop collapse test for a glycolipid surfactant (designated “BIF”) isolated from spent culture supernatant of P. aeruginosa. The surface tension of water results in dome-shaped and relatively tall water drops forming on a surface (No Add'n). Adding increasing amounts of this compound causes the water droplet to spread out and flatten into a pancake-like shape. The ability to collapse a water droplet is a characteristic of surfactants.

FIG. 6 is a bar graph demonstrating that a 24-hour old, pre-formed biofilm could be disrupted by the addition of partially purified supernatant in minimal salts medium; however, the minimal salts medium (M63) alone could not disrupt the biofilm. A pre-formed biofilm is either not removed, treated with M63 medium +Arg (arginine), treated with minimal M63 medium, or treated with M63 medium supplemented with partially purified supernatant and assayed for biofilm remaining after an additional four hour incubation. The addition of minimal medium with or without a source of carbon and energy (Arg) had no significant effect on biofilm dissolution. Partially purified supernatant in M63 medium efficiently dissolved the biofilm.

FIG. 7 is a bar graph demonstrating that hyperpiliated strains of Pseudomonas are resistant to the action of BIF. Bacterial strains were incubated in the presence of spent supernatants for 24 hours, and then the extent of biofilm formation was quantitated. The wild-type strain did not form a significant biofilm in the presence of BIF-containing spent supernatant, but the biofilm formed by the hyperpiliated strains (pilU and 33A9) were unaffected by the addition of BIF.

FIG. 8 is a photograph showing the ability of P. aeruginosa to form aggregates of cells at the air-medium interface. The addition of partially purified supernatant can completely disrupt these aggregates (the aggregates are indicated by arrows).

FIG. 9 is a bar graph demonstrating that partially purified supernatant is able to disrupt a biofilm even in the presence of the protein synthesis inhibitor Tc. Biofilms were grown for 24 hours, the medium was removed and replaced with partially purified supernatant (+ethanol, which is used to solubilize the Tc), partially purified supernatant+tetracycline, or M63 (as a positive control). The addition of Tc had no observable impact on the action of the supernatant; thus new protein synthesis is not required for the factors present in the supernatant to disrupt a biofilm.

FIG. 10 is a bar graph showing the ability of purified BIF to potentiate the activity of gentamycin (Gm). A biofilm of P. aeruginosa PA14 was pre-formed by growing the bacteria for 9 hrs in minimal M63 medium supplemented with arginine (0.4%) in 96 well dishes (100 μl of medium per well). After 9 hrs, half of the media was removed and replaced with fresh medium containing Gm (0.1 mg/ml final concentration), BIF (˜1 μM final concentration), or both compounds. The biofilm was quantitated after an additional 14 hrs of incubation.

FIG. 11A is a bar graph showing that S. aureus spent supernatant interferes with biofim development by S. aureus and P. aeruginosa. The AS550 value is an indirect measure of the extent of biofim formation.

FIG. 11B is a bar graph showing that the spent supernatant of S. aureus dissolves preformed biofims of S. aureus and P. aeruginosa. This data suggests that S. aureus produces a factor or factors that can disrupt biofilm formation, and that these factors act on both Gram-positive and Gram-negative organisms. The growth of the tester strains in the presence of spent supernatant or fresh medium was indistinguishable.

FIG. 12 shows R. etli biofilms formed on PVC under differing nutritional conditions.

FIG. 13 is a graph showing the growth curve of R. etli over a 48 hour time period. The top shows biofilm formation of R. etli on PVC over the same period of time.

FIG. 14 is a graph of the amount of R. etli biofilm formation over time.

FIG. 15 is a photograph showing crystal violet staining for R. etli biofilm formation on PVC at 19 and 20 hours.

FIG. 16 is a photograph showing biofilm formation over time for untreated controls versus cultures treated with R. etli spent culture supernatant.

FIG. 17 is a photograph showing biofilm formation after one of the following treatments: (1) no factor present; (2) factor present; (3) 1 hr 50 C.; (4) pronase; (5) protease XIII; and (6) 30 minutes of autoclaving.

FIG. 18 is a silver stained SDS polyacrylamide gel for a biofilm promoting activity purified from R. etli spent culture supernatant.

### DETAILED DESCRIPTION

Microorganisms are typically grown in the laboratory in either a closed culture system or a continuous culture system. In a closed system, growth occurs in a closed culture vessel containing a single batch of medium. The environmental conditions of a closed system change over time as nutrients are consumed and waste materials accumulate. In contrast, a continuous culture system maintains relatively constant environmental conditions by providing a continual flow of nutrients and removal of waste.

We have discovered that the growth of microorganisms in a closed or batch culture system, such as a microtiter dish, allows for the accumulation of microbially-produced factors, including biofilm modulating compounds. In a continuous flow through system, these compounds are continuously diluted away, making them difficult to detect. By harvesting and assaying the supernatants from closed culture systems, numerous compounds that are capable of affecting the biological activities of microorganisms can be identified and isolated.

The present invention provides methods for identifying compounds that have microbial biological activities, such as the ability to promote, inhibit or otherwise alter the formation of biofilms. The method involves obtaining supernatant from a closed culture system, exposing it to a target organism, and monitoring the effect of the exposure. The species of microorganism from which the supernatant is obtained (i.e., the source microorganism) may either be the same as or different from the target organism. The source microorganism and/or target organism may be a single species or multiple species of microorganisms.

The closed culture system of the invention preferably includes a closed vessel containing a single batch of medium that has been inoculated with at least one strain of microorganism. The culture vessel may be a microtiter dish, test tube, flask, or fermenter, or other suitable container. The vessel does not need to be sealed, covered, or enclosed, although it may be. The vessel may optionally be coated with various agents, such as, for example, serum, polysaccharides, bovine serum albumin, and surfactants. The environment of the closed culture system may be aerobic or anaerobic and may include any of wide variety of media, including, but not limited to, Luria-Bertani broth, trypticase soy broth, Todd-Hewitt broth, and M63 salts with MgSO₄, supplemented with GlcCAA, citrate and/or arginine.

The closed culture system of the invention may be a pure culture containing only one type of microorganism. Alternatively, the system may include a mixture of strains and/or species of microorganisms. The species may be known and characterized, unknown, or a mixture of known and unknown species. In certain embodiments, the closed culture system comprises raw environmental samples either from pristine environments (e.g., from soil, aquatic, rhizosphere; rhizoplane) or from human-impacted environments (toxic sites, industrial sites, agriculture, waste water treatment plants, etc.).

In a particularly preferred embodiment, the method of the invention employs multiple closed culture systems each of which contains a different type of microorganisms. For example, using a 96 or 364 or other well plate format it is possible to grow a different species of microorganism in each well, each producing a different supernatant for testing. This format allows for rapid screening of supernatants from many different species. Alternatively, each well may contain the same species of microorganism but provide a different set of environmental conditions, such as, for example, differing nutrient or media conditions. Using this format it is possible to quickly generate a wide variety of supernatants with differing properties for testing.

Identification of Compounds from Microbial Culture Supernatants that Inhibit Biofilm Formation or Disrupt Pre-formed Biofilms

In one embodiment, the method of the invention is used to identify compounds that are capable of inhibiting biofilm formation or disrupting pre-formed biofilms. The microorganisms of the closed culture system are grown to the desired phase of growth and the supernatant is then harvested. The supernatant may be harvested at any stage of microbial growth. In a preferred embodiment, the supernatant is harvested from microorganisms grown to the stationary phase. Alternatively, supernatants may be harvested from microorganisms at other stages of growth, including early-, mid-, and late-exponential phase. The supernatant is generally harvested by spinning the culture in a centrifuge to obtain a pellet of intact cells with an overlying layer of liquid (i.e., supernatant). Other methods that separate the fluid portion of the culture system from the cells may also be used, although it is not necessary to remove the cells from the supernatant prior to exposing the supernatant to the target organism.

Once the supernatant has been harvested, it is preferably filter-sterilized (0.2 micron filter) and then exposed to a target organism. Prior to exposure to the target organism, the supernatant may be diluted to ratios including, but not limited to, 1: 1, 1:2 and 1:4 with any suitable diluent. The supernatant may be purified and/or concentrated prior to dilution.

When testing for compounds that inhibit or prevent the formation of a biofilm, the supernatant is exposed to the target organism at the time of inoculation, or shortly thereafter. This is preferably accomplished by mixing the pure or diluted supernatant with fresh culture media in ratios varying from at least 1:1 to 1:10 (supernatant:medium). The mixture is then inoculated with the target organism, incubated for an appropriate period of time, and assayed for biofilm formation.

The exact incubation times will vary depending on the species of target bacteria that is being tested for biofilm formation. In general, when testing for compounds that inhibit biofilm formation, the target organism should be incubated for a period of time which would allow for biofilm formation under ordinary conditions. For example, if the target bacterial strain is Pseudomonas aeruginosa, it will preferably be assayed for biofilm formation about 8 hours after exposure to the supernatant. In the case of Staphyloccocal target strains, the assay will preferably be conducted >24 hours (generally 24-72 hours) following exposure to supernatant.

After exposure to the supernatant and appropriate incubation, the target organism is monitored to determine whether or not a biofilm develops. The target microorganism may be assayed for biofilm formation using standard assay techniques, such as crystal violet staining as described in WO 99/55368. In this assay, biofilm development is visualized in PVC wells by the addition of the dye crystal violet (CV), which stains the cells but not the plastic. The wells are incubated at room temperature for about 15 minutes to allow the CV to stain the surface attached cells and then thoroughly rinsed with water (to remove residual dye and unattached cells). CV-stained, surface-attached cells are quantified by solubilizing the dye in ethanol (or other organic solvents) and determining the absorbance at between about 550-600 nm. (For methods of staining and quantitating biofilms, see O'Toole, G. A. et al., 1999. Genetic approaches to the study of biofilms. Methods in Enzymology, R. Doyle (ed.) Academic Press, San Diego, Calif., 310:91-109). Supernatants that contain compounds capable of interfering with biofilm formation will generally have absorbance values lower than controls containing no supernatant. Absorbance values higher than untreated controls are indicative biofilm promoting activity. The sensitivity of this assay can be increased by using additional reporters for biofilm formation, which are known in the art.

When testing for compounds that disrupt pre-existing biofilms, the target organism is not exposed to the supernatant until after the target organism has formed a biofilm. The target organism biofilm is preferably formed using the biofilm formation assay described in WO 99/55368. Following incubation of the target organism and formation of the biofilm, the target organism is exposed to the supernatant. This is preferably accomplished by removing the media from the wells and replacing it with a mixture of fresh media and supernatant in ratios varying from at least 1:1 to 1:10 (supernatant:medium). After exposure to the supernatant, the biofilm is monitored for dissolution using, for example, the CV-staining assay method described above (other methods for measuring biofilm activity are described below). Supernatants that contain compounds with the ability to disrupt preformed biofilms will generally result in a lower absorbance reading as compared with controls containing no supernatant.

In addition to identifying compounds that disrupt biofilms and compounds that promote or inhibit biofilm formation, the present invention can also be used to identify compounds that are able to kill microorganisms in biofilm form. A biofilm is pre-formed and treated with a supernatant, extract, or purified preparation for a set period of time. After treatment, the supernatant is removed and replaced with fresh medium. The viability of the biofilm-grown cells can be assessed in two general fashions. First, after treatment and replacing the supernatant with fresh medium, the treated cells are allowed to outgrow. The planktonic population will be re-established only if there is a viable popoulation remaining in the biofilm. Thus, wells in which few or no viable cells are detected after outgrowth indicates that the biofilm-grown cells were susceptible to killing by factor(s) in the supernatant, extract, or purified preparation. Alternatively, after addition of fresh medium, the biofilm-grown cells can be removed from the surface, either by physical scraping or sonication (sound waves). (Oulahal-Lagsir, et al., 2000, Lett. Appl. Microbiol. January, 30(1):47-52; Raad et al., 1993, J. Infect Dis. Aug, 168(2):400-407; Tollefson, et al., 1987, Arch Surg. Jan, 122(1):38-43). After exposure of the target organism to the supernatant, viability can be tested using standard OD₆₀₀ measurements or plating of the target organism. Supernatants that result in OD₆₀₀ readings lower than a negative control containing no supernatant would be considered to have bacteriodical or bacteriostatic effects.

The method of the invention can also be used to identify compounds that act as “potentiators” of conventional antibiotics or biocides, i.e., compounds that increase the effectiveness of antibiotics or biocides against microorganisms growing in the biofilm form. In this assay, both spent supernatants and antibiotics/biocides are added to fresh medium in varying ratios as described above. The effect of the spent supernatants in combination with antibiotics/biocides on bacterial viability can be tested by standard OD₆₀₀ measurements and plating of the target bacteria.

A wide variety of organisms may be tested using the methods of the invention, both for the ability to produce activities and for the ability to be affected by an activity. Indeed, nearly any type of microorganism may be used to produce a supernatant for testing or as a target organism against which supernatants or extracts thereof are screened. The microorganisms may be archaea, bacteria, fungi, protozoa or algae. Example of bacterial organisms include, but are not limited to: Pseudomonas fluorescens, Pseudomonas aeruginosa, Pseudomonas acidovorans, Pseudomonas alcaligenes, Pseudomonas putida, Pseudomonas syringae, Pseudomonas aureofaciens, Pseudomonas fragi, Fusobacterium nucleatum, Treponema denticola, Porphyromonas gingivalis, Moraxella catarrhalis, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudo tuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Pasteurella multocida, Pasteurella haemolytica, Helicobacter pylori, Campylobacterfetus, Campylobacter jejuni, Campylobacter coli, Borrelia burgdorferi, Vibrio cholerae, Vibrio paramaemolyticus, Legionella pneumophila, Listeria monocytogenes, Neisseria gonorrhoeae, Neisseria meningitidis, Gardnerella vaginalis, Bacteroides spp., Clostridium difficile, Mycobacterium tuberculosis, Mycobacterium avium, Mycobacterium intracellulare, Mycrobacterium leprae, Corynebacterium diphtheriae, Corynebacterium ulcerans, Streptococcus spp., Enterococcus spp., Desulfvibrio spp., Actinomyces spp., Erwinia spp., Xanthomonas spp., Xylella spp., Clavibacter spp., Desulfomonas spp., Desulfovibrio spp., Desulfococcus spp., Desulfobacter spp., Desulfobulbus spp., Desulfosarcina spp., Deslfuromonas spp., Bacillus spp., Streptomyces spp., Clostridium spp., Rhodococcus spp., Thermatoga spp., Sphingomonas spp., Zymomonas spp., Micrococcus spp., Azotobacter spp., Norcardia spp., Brevibacterium spp., Alcaligenes spp., Microbispora spp., Micromonospora spp., Methylobacterium organophilum, Pseudomonas reptilivora, Pseudomonas carragienovora, Pseudomonas dentificans, Corynebacterium spp., Propionibacterium spp., Xanothomonas spp., Methylobacterium spp., Chromobacterium spp., Saccharopolyspora spp., Actinobacillus spp., Alteromonas spp., Aeronomonas spp., Agrobacterium tumefaciens, Staphylococcus aureus, Staphylococcus epidennidis, Staphylococcus hominis, Staphylococcus haemolyticus, Staphylococcus warneri, Staphylococcus cohnii, Staphylococcus saprophyticus, Staphylococcus capitis, Staphylococcus lugdunensis, Staphylococcus internedius, Staphylococcus hyicus, Staphylococcus saccharolyticus and Rhizobium spp., and mutant derivatives thereof.

In other embodiments of the present invention, the target organism or the microorganism from which supernatant is obtained may be a fungus, such as Absidia spp., Actinomadura madurae, Actinomyces spp., Allescheria boydii, Altemaria spp., Anthopsis deltoidea, Aphanomyces spp., Apophysomyces eleqans, Armillaria spp., Arnium leoporinum, Aspergillus spp., Aureobasidium pullulans, Basidiobolus ranarum, Bipolaris spp., Blastomyces dernatitidis, Botrytis spp., Candida spp., Centrospora spp., Cephalosporium spp., Ceratocystis spp., Chaetoconidium spp., Chaetomium spp., Cladosporium spp., Coccidioides immitis, Colletotrichum spp, Conidiobolus spp., Corynebacterium tenuis, Cryptoporiopsis spp., Cylindrocladium spp., Cryptococcus spp., Cunninghamella bertholletiae, Curvularia spp., Dactylaria spp., Diplodia spp., Epidermophyton spp., Epidermophyton floccosum, Exserophilum spp., Exophiala spp., Fonsecaea spp., Fulvia spp., Fusarium spp., Geotrichum spp., Guignardia spp., Helminthosporium spp., Histoplasma spp., Lecythophora spp., Macrophomina spp., Madurella spp., Magnaporthe spp., Malassezia furfur, Microsporum spp., Monilinia spp., Mucor spp., Mycocentrospora acerina, Nectria spp., Nocardia spp., Oospora spp., Ophiobolus spp., Paecilomyces spp., Paracoccidioides brasiliensis, Penicillium spp., Phaeosclera dematioides, Phaeoannellomyces spp., Phialemonium obovatum, Phialophora spp., Phlyctaena spp., Phoma spp., Phomopsis spp., Phymatotrichum spp., Phytophthora spp., Pythium spp., Piedraia hortai, Pneumocystis carinii, Puccinia spp., Pythium insidiosum, Rhinocladiella aquaspersa, Rhizomucor pusillus, Rhizoctonia spp., Rhizopus spp., Saccharomyces spp., Saksenaea vasifonnis, Sarcinomyces phaeomuriformis, Scerotium spp., Sclerotinia spp., Sphaerotheca spp., Sporothrix schenckii, Syncephalastrum racemosum, Taeniolella boppii, Taphrina spp., Thielaviopsis spp., Torulopsosis spp., Trichophyton spp., Trichosporon spp., Ulocladium chartarum, Ustilago spp., Venturia spp., Verticillium spp., Wangiella dermatitidis, Whetxelinia spp., Xylohypha spp., and their synonyms.

Alternatively, a part of a microorganism can be used as the target instead of the entire organism. Such parts could include, but are not limited to, an outer membrane protein or other surface adhesin.

A variety of parameters can be modified either in terms of assay conditions or the conditions under which the supernatants are prepared. For example, different environment conditions such as available nutrients, pH, incubation time, temperature, C source, osmolarity, etc., may lead to the presence of different compounds in the supernatant. They may also impact the effectiveness of an activity on a target organism.

A variety of indicators may be used in measuring biofilm activity, including, but not limited to, any of the following. Dyes or fluorescent indicators that stain microbial cells but not an abiotic surface like plastic (such indicators usually do not distinguish between viable and dead cells) such as, for example, crystal violet, safranin (used to stain Gram positive bacteria), and labeled lectins may be used. Specialized dyes that preferentially stain microbial cells compared to a biotic surface (such as mammalian cells) can also be used including, for example, calcoflour white (CFW), which preferentially stains yeast cells.

Alternatively, dyes or fluorescent indicators that bind nucleic acids, such as, DNA or RNA, including membrane-permeant or membrane-impermeant indicators may be used. Examples include Ethidium bromide, propidium iodide, DAPI, acridine orange, Hoechst dyes, and their derivatives.

In addition, dyes or fluorescent indicators that are sensitive to cellular growth or metabolic parameters may be used to distinguish between live and dead cells. Such parameters include cell membrane permeability, cell membrane potential, enzymatic activity, oxidation-reduction potential, sugar utilization and measurement of cellular ATP levels. Examples of such indicators include rhodamine 123, fluorescein diacetate (FDA), alkaline phosphatase, Alamar Blue (Accumed, Westlake Ohio, USA), Syto-9 (Molecular Probes), FUN-1 (Molecular Probes), tetrazolium salts such as 3-[4,5, dimethylthiazol-2-Y1]-2,5-diphenyltetrazolium bromide (MTT) and 5-Cyano-2,3-ditolyltetrazolium chloride (CTC), beta-galactosidase (lacZ), and Ethidium Bromide (EtBr).

Reporters such as green fluorescent protein (GFP), including derivatives with altered excitation/emission spectra and half-lives, β-galactosidase(lacZ), chloramphenicol transacylase (CAT), luciferase (luc), bacterial bioluminescence reporters (lux) and reporters that are differentially expressed (up or downregulated) or exclusively expressed in microbes in the biofilm or planktonic form may be used. These reporter genes may be placed under the control of a variety of different promoters that may be constitutively expressed (including promoters for ribosomal RNA genes such as 16s and 23s rRNA), specifically expressed under defined environmental conditions, or expressed differentially or exclusively in the biofilm form. Other indicators include, but are not limited to, RNA and DNA probes such as those derived from 16s and 23s rRNA, or those derived from biofilm-specific markers, which may be indicator tagged (fluorophores, radiolabels, enzymes, or antigens); mono or polyclonal antibodies (tagged with fluorophores, radiolabels, or enzymes) against microbial antigens; and radioisotopes.

The above indicators may be used in a variety of assay methods to measure biofilm activity. These assay methods include: qualitative visual observations; bacterial enumeration assays, including viable plate counts, measurements of dry weights of cells, and growth in liquid cultures; spectophotometric measurements, including UV visible, chemiluminscence and fluorescence assays; immunological methods including ELISA assays; microscopic examinations, including phase contrast, epifluorescence, deconvolution fluorescence microscopy, electron microcopy (scanning and transmission), confocal laser microscopy and photon counting microscopy; in situ hybridization techniques including fluorescent in situ hybridization (FISH); flow cytometry; direct measurement of microbial physiological parameters, including sugar uptake, pH, ion fluxes, membrane potential, and oxygen tension including the use of instruments such as a microphysiometer; and assays based on the detection of surface-associated or secreted microbial factors, including toxins such as exotoxinA and exoenzyme S in P. aeruginosa. (Korber, D. R., et al., 1999, Reporter system for microscopic analysis of biofilm bacteria in Methods in Enzymology “Biofilms”, Ron J. Doyle ed. v310:3-20; and Bjarke Bak Christensen, et al., 1999, ibid. 20-43).

Using the methods of the invention, we have identified activities in spent culture supernatants that are capable of interfering with biofilm formation and causing dissolution of preformed biofilms. These anti-biofilm activities are able to impact and hinder various aspects of biofilm development without necessarily affecting the viability or growth of the organism. The purification and characterization of one of these activities is described below in Example 3. The identification of this activity illustrates that supernatants from closed culture systems may be used as a means for inhibiting biofilm development as well as disrupting preformed biofilms. In addition, the supernatants may be purified to increase the concentration of biofilm modulating compounds. This increases the effectiveness of the supernatants as biofilm modulating substances. The concentration of biofilm modulating substances may be increased using any of a wide variety of well-known purification and isolation techniques. Alternatively, the biofilm modulating compounds may be chemically synthesized using methods known in the art.

Glycolipid Compound Capable of Modulating Biofilm Formation

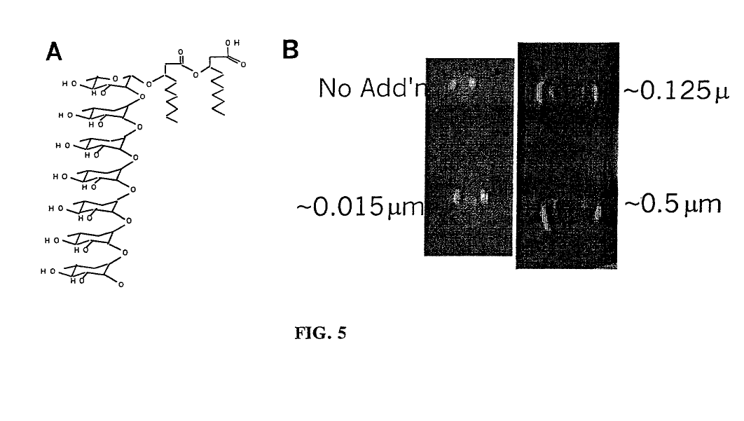

The invention also features a novel compound that was isolated from the spent culture supernatant of P. aeruginosa. This compound is a glycolipid surfactant that is able to interfere with biofilm development in P. aeruginosa and other organisms. The compound is predicted to have a chain of at least three rhamnose moieties, preferably seven rhamnose moieties, linked to a lipid moiety. An embodiment of this compound is shown in FIG. 5A.

This compound can be isolated by growing P. aeruginosa for 36-48 hours (stationary phase) in a closed culture system, preferably in a minimal salts (M63) medium, supplemented with glucose (0.2%), casamino acids (0.5%), and iron (μm concentrations). The spent supernatant is harvested and boiled for about 30 minutes, filtered through a 0.45 μm membrane, and fractionated by low-pressure chromatography using a hydrophobic interaction matrix (C18 resin) followed by ion exchange chromatography (DEAE sephadex A25 resin). The compound elutes from the C18 column in a broad peak between 50-100% acetonitrile and from the DEAE column in a broad peak between 0.5 and 1 M NaCl. Reverse phase HPLC is the final step in the purification yielding a single peak at ˜95% acetonitrile.

This compound is capable of inhibiting the biofilm formation, when exposed to cells of the target organism at concentrations between about 1-10 μm. The novel biological surfactant of the invention can potentially be used to eliminate or prevent biofilm formation in a variety of clinical and industrial settings.

The present invention is illustrated by the following examples, which are in no way intended to be limiting of the invention.

### EXAMPLE 1

### Assay System for Biofilm Development.

Biofilms were formed using the assay system described in WO 99/55368, which is based on the ability of bacteria to form biofilms on polyvinylchloride plastic (PVC), a material which is used to make catheter lines (Lopez-Lopez, G., et al., 1991, J. Med. Microbiol. 34: 349-353). Biofilm formation was assayed by the ability of cells to adhere to the wells of 96-well microtiter dishes made of PVC (Falcon 3911 Microtest III Flexible Assay Plate, Becton Dickinson Labware, Oxnard, Calif.) using a modification of a reported protocol (Fletcher, M., 1977, Can. J. Microbiol. 23: 1-6). The appropriate medium was inoculated with microbial cells, added to microtiter dish wells (100 μL/well), and incubated at between 25° C. to 37° C. to allow the cells to grow and form biofilms on the walls of the microtiter dish. The incubation times and media conditions were varied depending on the species of microorganism being cultured (8-48 hours for P. aeruginosa, P. fluorescens, and E. Coli; 36-48 hours for Staphylococcus aureus, Streptococcus mutans; Streptococcus sanguis, and Streptococcus gordonii). The following table provides the media conditions that promoted formation of biofilms.

TABLE 1
Assay conditions that promote biofilm formation in 96 well dishes

| | Microbe | | | | |
|---|---|---|---|---|---|
| | P. | E. | P. | S. | S. |
| Media | aeruginosa⁴ | coli⁴ | fluorescens⁴ | aureus⁴ | mutans^{4,5} |
| GlcCAA¹ | +, Standard | + | + | − | − |
| | Conditions⁶ | | | | |
| Citrate¹ | + | NT | + | − | − |
| Arginine¹ | + | NT | + | − | − |
| Arginine + | − | NT | − | NT | NT |
| Hi Osmolarity² | | | | | |
| LB³ | + | + | + | + | + |
| TSB³ | NT⁷ | NT | NT | +⁶ | NT |
| THB + Glc³ | NT | NT | NT | + | + |

“+” indicates that the conditions promoted biofilm formation
¹The base medium was M63 salts with MgSO₄ (1 mM) and supplemented with GlcCAA (glucose, 0.2%; casamino acids, 0.5%), Citrate (O.4%), or Arginine (0.4%).
²Hi osmolarity refers to >0.2 M NaCl or >10% sucrose.
³LB = Luria-Bertania broth, TSB = trypticase soy broth, THB + Glc = Todd-Hewitt broth + 0.2% glucose.
⁴The phenotype of the biofilm formed by these organisms is as follows:
1. P. aeruginosa, P. fluorescens- A ring forms at the air/medium interface, with the exception of arginine which yields a complete coating of the well. Biofilm formed after incubation for 8-48 hrs at RT-37° C. with no agitation. Detachment of biofilm cells was observed by 48 hours.
2. E. coli - Biofilm completely coats well. Biofilm forms after incubation for 8-48 hrs at RT-37° C. with no agitation.
3. Staphylococcus aureus, Streptococcus mutans/sanguis/gordonii - Biofilm formed at the bottom or side of the well after incubation for 36-48 hrs at 37° C. with no agitation.
⁵Growth requires a low oxygen environment. (These conditions are also optimal for growth of S. gordonii and S. sanguis)
⁶Can detect BIF-like activity when cells are grown in wells.
⁷NT = not tested

Biofilm formation was monitored visually by the addition of the dye crystal violet (CV). This purple dye stains the bacterial cells, but does not stain plastics such as PVC. 125 μL of a 1% solution of CV was added to each well. The CV-stained plates were then incubated at room temperature for about 15 min, rinsed thoroughly with water (to remove residual dye and cells which are not firmly attached to the surface), and scored for the formation of a biofilm. Under standard assay conditions (M63 salts with MgSO₄, (1 mM) supplemented with GlcCAA) P. aeruginosa biofilms formed at the interface between the air and liquid medium after incubation for 8-48 hours with no agitation. This observation was consistent with the fact that, under these growth conditions, oxygen is the primary electron acceptor available. An uninoculated medium control did not form the characteristic attachment ring.

For P. aeruginosa, phase-contrast microscopy was used to monitor both the initial formation of a monolayer of cells and the subsequent development of microcolonies in the wells of the microtiter dishes. These microcolonies are the precursors of the complex architecture that is a hallmark of biofilm development. We also observed that, after extended incubation, P. aeruginosa would begin to detach from the PVC plastic. This detachment may be in response to a lack of fresh nutrients or factors produced by the microorganisms, indicating that the microtiter dish assay can also be used to explore the mechanisms of biofilm detachment. Thus, the PVC microtiter dish assay, which employs a batch culture approach, is a very useful system to analyze biofilm development, because of the ability to study biofilms on medically relevant surfaces and the fact that both attachment and detachment, as well as various properties and phenotypes associate with biofilms, can be monitored in a high throughput system.

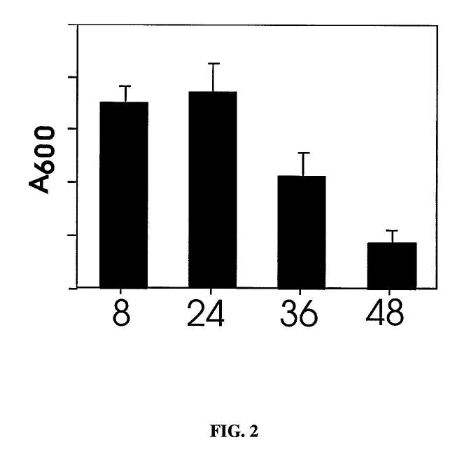

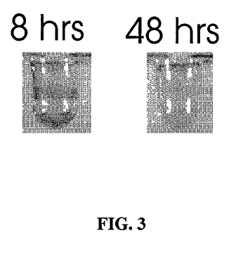

The microtiter dish method also provides semi-quantitative information on the relative rate and extent of biofilm formation by the wild-type (wt) and mutant strains. Biofilm formation was quantified by the addition of 200 μL of 95% ethanol (or other organic solvent) to each CV-stained microtiter dish well (ethanol solubilizes the dye), of which 125 μL was subsequently transferred to a new microtiter dish and the absorbance at or near 600 nm was determined. FIG. 2 shows the quantitation of biofilm formation over the course of 48 hrs. The A₆₀₀ value represents the relative extent of biofilm formation over the 48 hr incubation period. FIG. 3 shows the wells from an 8 hr and 48 hr old biofilm of P. aeruginosa. As demonstrated in these figures, the extent of biofilm formation over time initially increases, and then decreases.

We found that growing P. aeruginosa on arginine serves as the best conditions for assaying this organism because it promotes the best biofilm (and best signal-to-noise ratio for assays). We also discovered that bacteria grown under these conditions acquire the antibiotic and detergent resistance typically associated with flow cell-grown biofilms.

### EXAMPLE 2

### Identification of an Extracellular Activity that Inhibits Bioflim Formation.

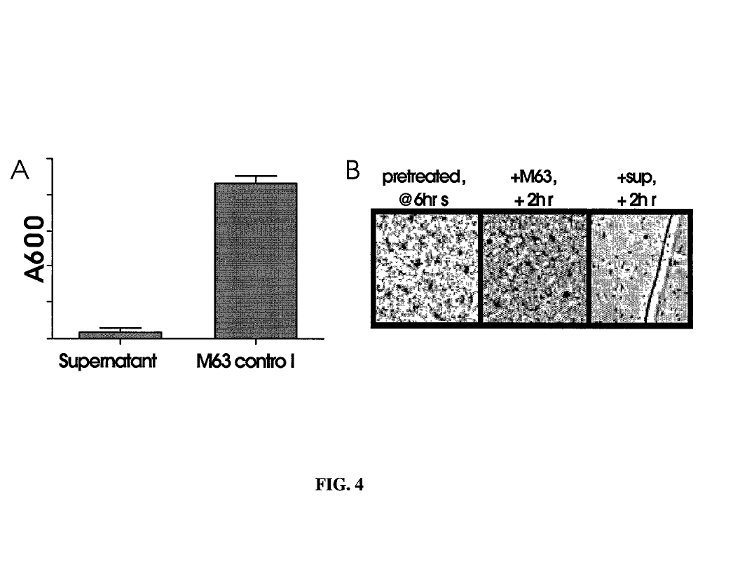

The decrease in biofilm formation at 48 hours shown in FIGS. 2 and 3, was indicative of a mechanism by which cells can detach from a surface. We analyzed the spent culture supernatant of P. aeruginosa PA14 to identify the presence of an extracellular factor capable of interfering with biofilm development. A freshly inoculated culture of P. aeruginosa was mixed with either spent supernatant from cultures of P. aeruginosa (as prepared in example 1) or minimal M63 medium (control). As shown in FIG. 4A, when spent supernatants were added to the freshly inoculated culture, no observable biofilm was formed. However, the addition of spent supernatant did not affect the growth of the wild type strain. This demonstrates that spent supernatants of P. aeruginosa contain an activity, which we have designated “BIF”, that can inhibit the formation of biofilms. Since the BIF activity is found in the supernatants, there is presently no reason to believe that BIF is tightly associated with the cell surface.

Our experiments also indicate that partially purified spent supernatants can disrupt a pre-formed biofilm. Biofilms were allowed to develop for 6 hrs on plastic (FIG. 4B, left panel) then the medium was replaced with either fresh medium (M63) or spent supernatant, incubated for an additional 2 hrs, rinsed, and the biofilm assessed by phase-contrast microscopy. Treatment with fresh medium resulted in no effect on the biofilm (FIG. 4B, center panel). Exposing the biofilm to spent supernatant caused a marked decrease in the number of cells in the biofilm (FIG. 4B, right panel), however a dispersed monolayer of cells did remain attached to the plastic surface. This data indicates that the spent supernatant disrupts cell-to-cell interactions and can both speed the dissolution of a biofilm and prevent the initial formation of a biofilm.

Continued purification of BIF suggests that more than one compound may be responsible for the activities describe above. In particular, spent supernatants of P. aeruginosa PA-14 appear to contain two distinct activities. BIF, a rhamnolipid, can interfere with the formation of a biofilm. A second factor, which is uncharacterized, can both inhibit biofilm formation and disrupt pre-formed biofilms. These two factors can be separated by size fractionation. BIF is retained when dialyzed against a 500 MWCO dialysis membrane, while the second inhibition/dissolution factor is lost upon dialysis versus this membrane. Both activities are resistant to boiling for 30 minutes.

We have harvested supernatants at various times after inoculation of the cultures. Using a variety of media, including LB and minimal medium with glucose or arginine, we do not detect BIF activity until ˜24 hrs of incubation. These data suggest that the synthesis of BIF occurs in starved cells or cells of high cell density.

### EXAMPLE 3

### Characterization and Purification of BIF.

We have developed a method for the purification of BIF and have determined a tentative structure for this compound. Supernatants of P. aeruginosa were boiled for 30 minutes, filtered through a 0.45 μm membrane, and fractionated by low-pressure chromatography using a hydrophobic interaction matrix (C18 resin) followed by ion exchange chromatography (DEAE sephadex A25 resin). The BIF activity eluted from the C18 column in a broad peak between 50-100% acetonitrile and from the DEAE column in a broad peak between 0.5 and 1 M NaCl. Reverse phase HPLC is the final step in the purification yielding a single peak with BIF activity at ˜95% acetonitrile +0.05 trifluoroacetic acid. This peak was subjected to both mass spectrometric and NMR analysis and based on these analyses we determined the predicted structure of the novel glycolipid surfactant shown in FIG. 5A. The compound is composed of a chain of 7 rhamnose moieties linked to a lipid. One of the properties of surfactants is their ability to reduce the surface tension of water, resulting in the “collapse” of water droplets. FIG. 5B shows that adding increasing amounts of purified BIF causes the collapse and spreading of the water droplets consistent with its identification as a surfactant-like molecule.

### EXAMPLE 4

### Dissolution of a Preexisting Biofilm.

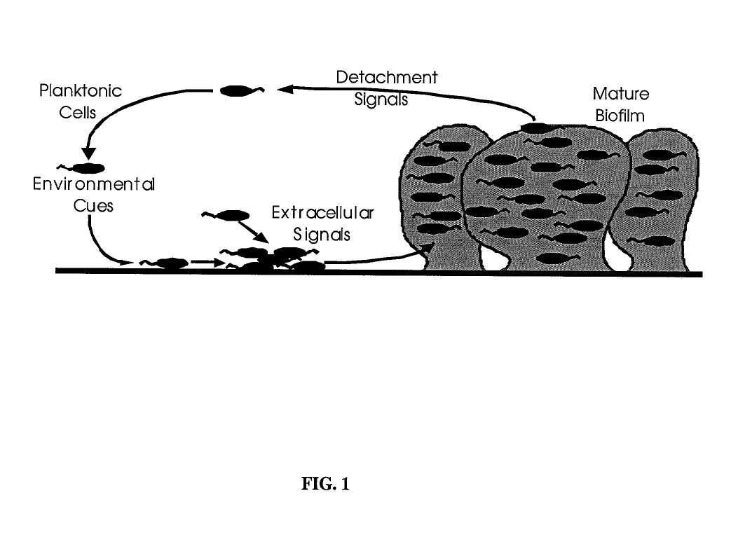

Over the course of 48 hrs in our microtiter dish system, a biofilm forms (reaching a maximum at ˜24 hrs) and then cells are released from the biofilm. This ability to detach after extended incubation suggested that, like attachment, detachment might be part of the normal biofilm development (See FIG. 2). We have purified and characterized an activity in the spent supernatants of P. aeruginosa that prevents this same strain from forming a biofilm and have partially purified an activity that both inhibits P. aeruginosa from forming a biofilm and disrupts a pre-formed biofilm of the same strain. Our hypothesis is that this extracellular activity plays a role in detachment of cells from a surface and, therefore, promotes the biofilm-to-planktonic cell transition (see FIG. 1). This factoreither inhibit attachment or speed detachment of cells or both.

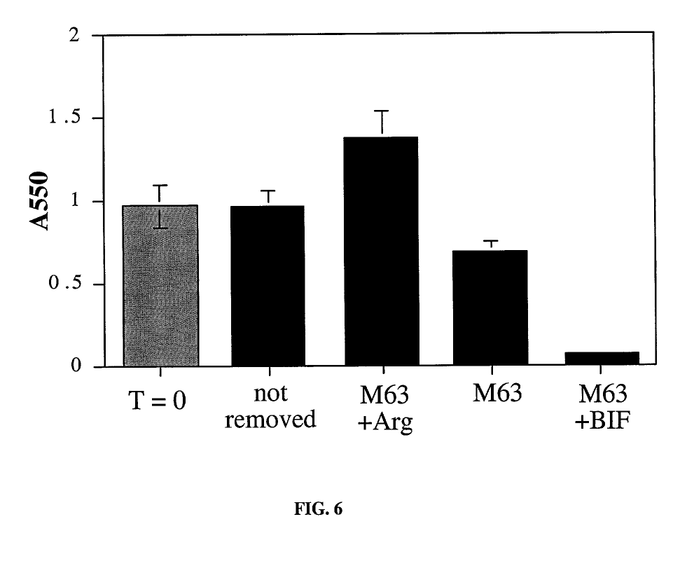

We performed experiments that indicate that a factor in partially purified spent supernatants plays a role in the release of cells from the biofilm (we refer to this phenomenon of cell release as “dissolution”, although some cells may remain attached after treatment with supernatant). A pre-formed biofilm was either not removed, or treated with M63 medium +Arg (arginine), treated with minimal M63 medium or treated with M63 medium supplemented with partially purified supernatant, and assayed for biofilm remaining after an additional four hour incubation. As shown in FIG. 6, a 24-hour old pre-formed biofilm was disrupted by the addition of partially purified supernatant in minimal salts medium; however, the minimal salts medium (M63) alone did not disrupt the biofilm. The inability of minimal salts medium to disrupt a pre-formed biofilm indicates that starvation is not sufficient to trigger detachment.

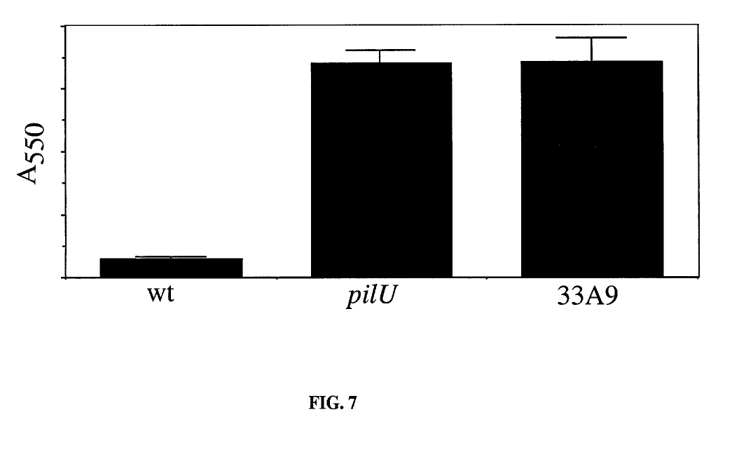

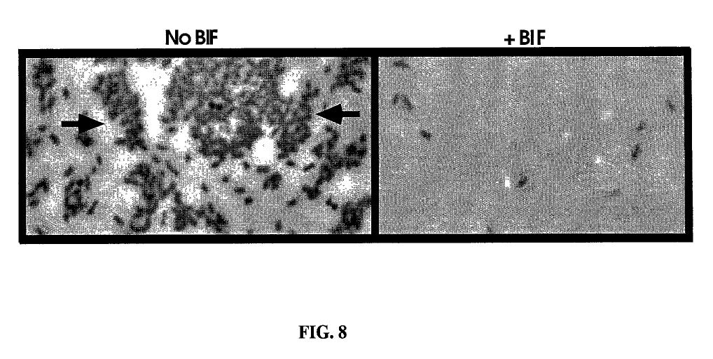

It is not clear if the effect of BIF on biofilms is mediated via gene regulation or by physical effects that disrupt cell-to-cell and/or cell-to-surface interactions (these two modes of action are not mutually exclusive). We have several lines of evidence consistent with the role of supernatants physically interfering with biofilm stability, and in particular, interfering with cell-to-cell interactions. First, supernatants disrupted the microcolonies of an already formed biofilm, but left a monolayer of cells intact (FIG. 4B). We have also identified two strains, pilU216 and 33A9, that make BIF but are resistant to its activity, and both strains are hyper-piliated (FIG. 7). Pili are thought to play an important role in cell-to-cell interactions in other microbes. As judged by phase-contrast microscopy, BIF also appears to prevent formation of the aggregates of wt cells that are often formed in cultures at the air-medium interface.

Our best estimations of BIF concentration suggest that relatively high levels (˜1-10 μM) are required for activity, which is also consistent with physical interruption of cell-to-cell interactions as the mechanism of BIF action. We believe that supernatants can physically disrupt pre-formed cell-to-cell interactions (thereby dissolving an already formed biofilm) and/or prevent the formation of these cell-to-cell interactions.

Disruption of the Biofilm by Supernatant does not Appear to be Dependent on Protein Synthesis.

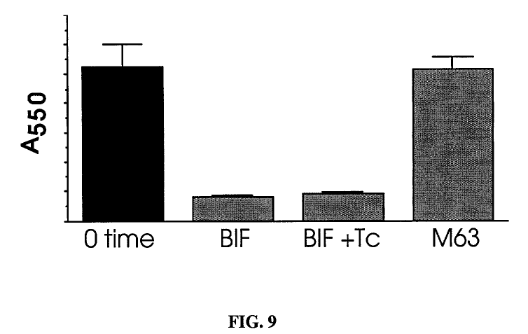

As shown in FIG. 4B, it is possible to pre-form a biofilm, then disrupt the biofilm in less than two hours by the addition of partially purified spent supernatant. Using this assay, the effect of protein synthesis inhibitors (i.e., tetracycline, Tc) on the supernatant-mediated disruption of a pre-formed biofilm was tested. Protein synthesis and transcription inhibitors should have no effect on the biofilm disruption activity if the factors present in the supernatant act solely through a physical disruption of the biofilm. Biofilms were grown for 24 hours, the medium was removed and replaced with spent supernatant (+ethanol, which is used to solubilize the Tc), spent supernatant +tetracycline, or M63 (as a positive control). As shown in FIG. 9, spent supernatant was still able to disrupt a biofilm even in the presence of the protein synthesis inhibitor Tc.

### EXAMPLE 5

### Biofilm Interfering Activity in Other Organisms.

As described above, an activity was isolated from the spent culture supernatants of P. aeruginosa PA14. The experiments described above demonstrate the ability of partially purified supernatant to block the formation and cause the dissolution of P. aeruginosa PA14 biofilms. We have also shown that BIF interferes with biofilm formation in other organisms, including E. coli K12 and Pseudomonas fluorescens WCS365, as well as a number of clinical isolates of P. aeruginosa. BIF also appears to have bactericidal activity towards Staphylococcus aureus Newman.

In addition, we have assayed other bacteria for their ability to produce extracellular factors that interfere with biofilm development. The table below summarizes the results of these data.

TABLE 2
Summary of bioflim modulating activity in various microorganisms.

| Strain | Activity vs. Producer? | Activity vs. P.a. PA14? |
|---|---|---|
| P. aeruginosa PA14 | Yes | Yes |
| P. fluorescens | Yes | Yes |
| WCS365 | | |
| E. coli K12 | Yes | NT |
| S. aureus Newman | Yes | Yes |
| S. mutans | NT | Yes |

NT = Not tested.

As shown in the table above, each of the organisms tested produced a biofilm modulating activity that was effective not only against the strain which produced the activity, but also against P. aeruginosa PA14.

We have set-up conditions using 96 well plates that allow us to screen for mutants defective in production of biofilm modulating activities using S. aureus as a model. S. aureus was incubated in 96 well plates for 48 hrs in TSB medium. 50 μL of the supernatant was then transferred to a new well containing 50 μL of M63 medium supplemented with MgSO₄ and arginine (this medium had been pre-inoculated with P. aeruginosa). After 8-24 hrs of incubation, biofilm formation was assayed as described above. Results showed that spent supernatants of S. aureus inhibited biofilms of P. aeruginosa (as well as S. aureus). Thus P. aeruginosa (which is easier to grow and grows more quickly than S. aureus) can act is an indicator strain to track biofilm modulating activities produced by S. aureus. P. aeruginosa can also be used to assay the production of biofilm modulating activities produced by S. mutans. Therefore this assay has utility as a method to generate biofilm modulating activities in an easy to assay format and/or rapidly screen these activities versus a variety of microbes.

### EXAMPLE 6

### BIF Potentiates the Effects of Antimicrobial Compounds on Biofilm-Grown Cells.

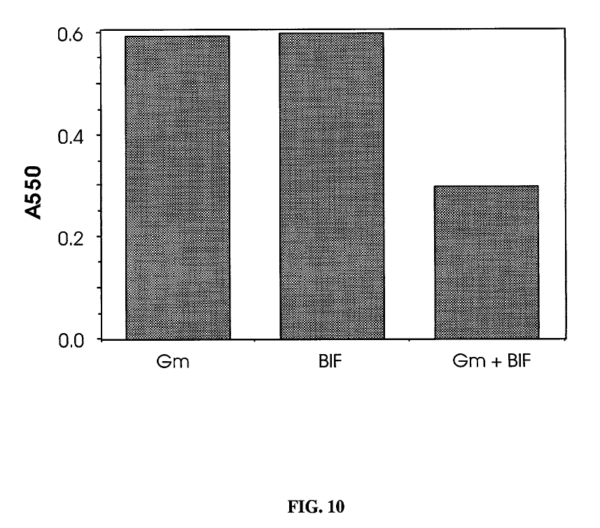

We predicted that addition of purified BIF might potentiate the effects of antimicrobial compounds. To test this hypothesis, we pre-formed a biofilm of P. aeruginosa by growing the bacteria for 9 hrs by growth in minimal M63 medium supplemented with arginine (0.4%) in 96 well dishes. The preformed biofilms were then treated with BIF alone or BIF plus gentamycin (Gm) at 0.1 mg/ml final concentration. At the concentrations tested, BIF (˜1 μM) and Gm (0.1 mg/ml) had no effect on pre-formed biofilms of P. aeruginosa when these compounds were used individually (FIG. 10). However, when applied simultaneously, after 14 hours of incubation, almost half the biofilm was eliminated. This data is consistent with BIF potentiating the effects of antibiotics on biofilm grown cells.

### EXAMPLE 7

### Identification of Two Extracellular Factors from S. aureus Newman that Interfere with Bioflim Development.

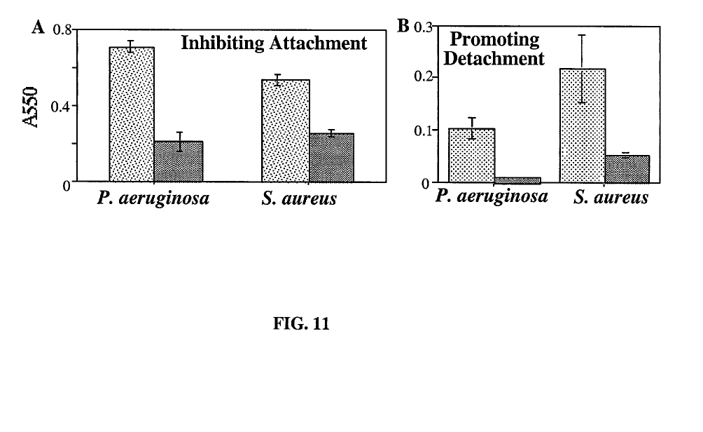

Our experiences with the Gram-negative bacterium P. aeruginosa, suggested that bacteria could produce extracellular compounds which could interfere with their own biofilm formation pathways, as well as biofilm formation by other microorganisms. Based on these observations, we asked whether Gram-positive bacteria could produce similar activities. We began these studies using S. aureus as a model organism. Using the 96 well dish system described above, we have shown that spent supernatant of S. aureus Newman contain at least two distinct activities (see FIG. 11) that can interfere with the development of biofilms by this and other organisms. The assay used to detect these activities is a variation of that described above where biofilms are detected in 96 well dishes using the CV stain.

To assay for inhibition of initial attachment, spent supernatants or fractions from purification steps were mixed 1:1 with freshly inoculated culture of the “tester” strain (i.e. target microorganism). The “tester” strain was typically either S. aureus or P. aeruginosa. Without the addition of crude or fractionated spent supernatants, these organisms will typically form a biofilm under our standard assay conditions after 48 (S. aureus) or 8 hrs (P. aeruginosa). Using this assay system, we found that spent supernatants of S. aureus Newman (48 hr. old cultures) interfere with the formation of biofilms by both S. aureus and P. aeruginosa (FIG. 11A). The advantage of P. aeruginosa as a tester strain is that it considerably speeds the pace at which we can follow biofilm inhibition activity and thus purify the compound(s) (as described in detail below).

To assay detachment activity, a biofilm of S. aureus and P. aeruginosa was allowed to pre-form for 48 to 24 hrs, respectively. The medium was removed from the well and replaced with spent supernatants or fractionated supernatants, incubated additional 4-8 hrs, and assayed for the formation of the biofilm. Spent supernatants of S. aureus were able to dissolve the pre-formed biofilms of both S. aureus and P. aeruginosa (FIG. 11B). As a control, the growth or viable cell counts of bacteria in each treated well can be assayed to determine if any component of the spent supernatant is inhibiting growth of, or killing, the tester strain. These preliminary data suggest that the activities present in spent supernatants of S. aureus have a broad effect on biofilms formed by both Gram positive and Gram negative organisms.

We originally designated the biofilm interference activity found in spent supernatants of S. aureus Newman as NIF, for S. aureus Newman Interference Factor. As shown in FIGS. 11A and 11B, spent supernatants from this organism could both block initial biofilm formation and dissolve a preformed biofilm. Additional studies generated two lines of evidence that supported the existence of two discrete biofilm-interfering activities in spent supernatants of this S. aureus Neuman. First dialysis of the spent supernatants using a 500 molecular weight cutoff (mwco) membrane results in loss of activity which dissolves a pre-formed biofilm, but this dialyzed supernatant can still block initial attachment. The two factors present in spent supernatants of S. aureus have been designated NIF-A and NIF-D. NIF-A is defined as a compound that can block the initial attachment of bacteria to a surface, while NIF-D blocks initial biofilm formation and promotes detachment of a preformed biofilm. The dialysis results described above suggest that NIF-D is less than 500 mw, while NIF-A is greater than 500 mw. Furthermore, both activities are lost by dialysis using a 6000-8000 mwco membrane, suggesting that NIF-A is between 500 and 8000 mw.

A second line of evidence suggested that there are two, distinct biofilm interference factors in spent supernatants of S. aureus Newman. In order to purify and enrich the biofilm interference factors, we began a series of fractionation experiments. Hydrophobic interaction chromatography (C18 resin developed with a step gradient of 0, 50%, and 100% acetonitrile) resulted in NIF-D activity being detected in the flow-through fraction while NIF-A activity eluted at 50% acetonitrile, suggesting that NIF-A has more hydrophobic character than NIF-D. This fractionation step clearly demonstrated that NIF-A and NIF-D activities could be separated. Taken together, the dialysis experiments and hydrophobic interaction chromatography are consist with the presence of at least two biofilm interference factors in spent supernatants of S. aureus Newman.

Interestingly, like the BIF activity described above, biofilm interference activity produced by S. aureus is resistant to boiling, not affected by treatment with proteases, and can only be detected in the supernatant after extended growth of the cultures (24-48 hrs). A summary of NIF-A and NIF-D activities is shown in the Table below.

TABLE 3
Summary of NIF Activities.

| Assay/Treatment/Properties | NIF-A | NIF-D |
|---|---|---|
| Inhibit Biofilm Attachment | Yes | Yes |
| Promote Bioflim Detachment | No | Yes |
| Resistant to Boiling | Yes | Yes |
| Resistant to Protease Treatment | Yes | Yes |
| Binds to C18 resin | Yes | No |
| Estimate Molecular Weight (Da) | 500-8000 | <500 |

Partial Purification of the NIF Activities.

We have developed fractionation techniques to purify NIF-A and NIF-D. Spent supernatants were prepared from cells grown in LB for 48 hrs. The bacteria were removed by centrifugation, boiled in a water bath for 30 minutes, and then filtered through a 0.22 μm filter to remove particulate matter and sterilize the supernatant. This spent supernatant can be stored at 4° C. until used. We selected a hydrophobic interaction resin (C18) as the first means of fractionating the supernatant. The column was developed with a step gradient of 0, 50, and 100% acetonitrile (ACN), and as described above, NIF-A activity eluted at 50% ACN while NIF-D activity was detected in the flow-through fraction. These two activities can be distinguished based on their activities (blocking attachment and/or promoting dissolution of a biofilm) and their size (by dialysis). The ability to resolve NIF-A and NIF-D is an important result because it provides an easy means to separate these two activities in the first fractionation step.

### EXAMPLE 8

### Candida Albicans SC5314 Biofilm Disruption by Pseudomonas aeruginosa PA 14.

Experiments with Candida albicans demonstrated that compounds produced by bacteria can also modulate biofilms produced by eukaryotic organisms. In these experiments, Candida albicans SC5314 biofilms were pre-formed on 16 mm×150 mm borosilicate glass tubes (Bellco, Vineland, N.J.) by inoculating M63 medium +0.2% glucose with yeast-form Candida albicans, and then incubating at 37° C. with shaking on a Rollerdrum. Pseudomonas aerugiinosa PA-14 was grown in the same medium to a density of greater than OD>1 at 600 nm.

After biofilms were formed, spent medium and suspended C. albicans cells were removed from the tube, and replaced with an equal volume of either stationary phase PA14 culture (grown in M63+glucose at 37° C.), fresh M63 medium, or C. albicans culture. Biofilms were again incubated at 37° C. with shaking after the medium exchange.

Addition of stationary phase PA14 cultures to tubes with pre-formed SC5314 biofilms lead to the disruption of the C. albicans biofilms in less than 1 hour. Similarly, PA14 cultures diluted 5-fold in fresh M63 medium retained biofilm-disrupting activity. After addition of PA14, C. albicans did not reattach to the glass tubes over the next 48 hours. SC5314 biofilms in tubes that received fresh M63+glucose or stationary phase C. albicans cultures remained intact.

Both vital staining techniques and plate count enumeration suggests that viability of C. albicans is not significantly affected during this time. Since P. aeruginosa do not attach to the filaments at a time period of<1 hour, the observation that biofilms detached within this time frame indicates that detachment is likely due to a secreted factor in the PA14 supernatant.

### EXAMPLE 9

### Identification of Bioflim Promoting Factors.

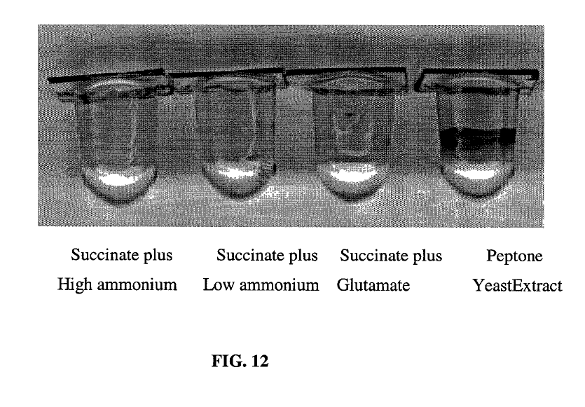

Not all bacteria proceed through the same developmental cycle of biofilm formation as that described above. In particular, we discovered that the symbiotic nitrogen fixing bacterium Rhizobium etli, in contrast to P. aeruginosa, forms dramatically different biofilms on PVC depending on the nutritional conditions (See FIG. 12).

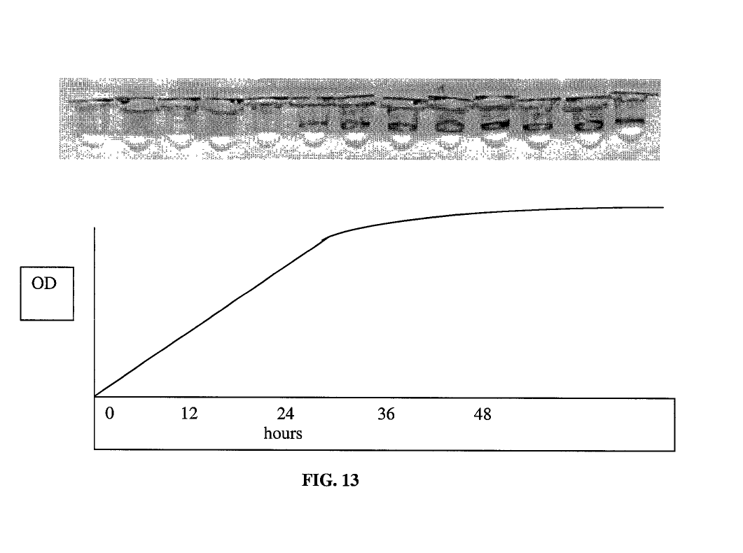

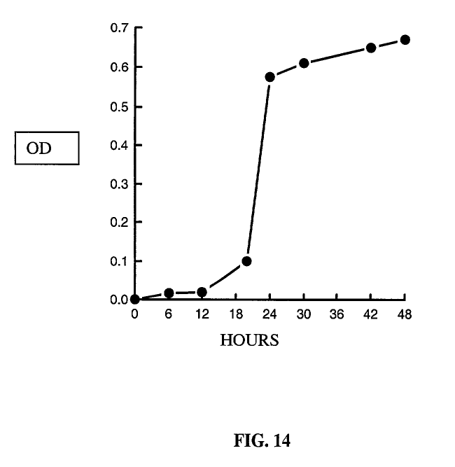

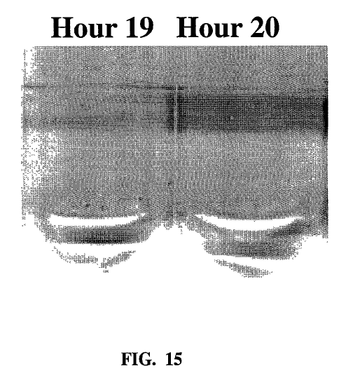

Most importantly, we found that the biofilms of R. etli form on PVC plastic with remarkably different kinetics than P. aeruginosa. Instead of the formation process taking making hours along the entire duration of exponential growth, R. etli biofilms form very rapidly at the onset of stationary phase. (See FIG. 13). Indeed, biofilm formation is very fast and happens within a period of one to two hours at the entry into stationary phase. The quantitative results of the amount of biofilm formation over time are shown in FIG. 14. The crystal violet staining for the period between 19 and 20 hours is shown in FIG. 15.

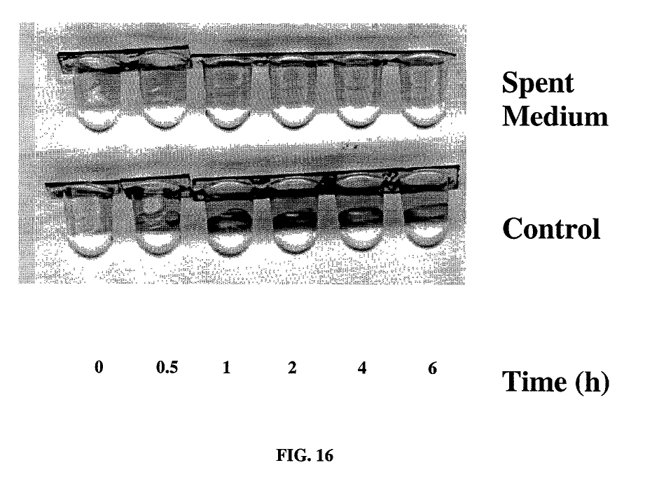

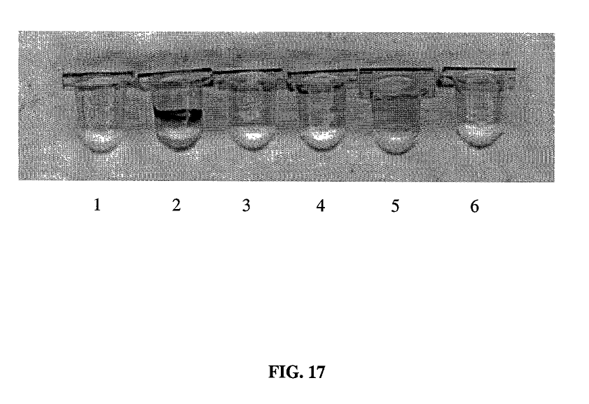

These results indicated that there was a soluble factor in the supernatants of stationary phase cultures that could stimulate the formation of biofilms. To test this hypothesis, we took a culture of R. etli that was in mid-exponential phase and added to it conditioned medium from a stationary phase culture of the same bacterium. The results are shown in FIG. 16. On the bottom are the control wells that contained the exponential phase cultures untreated for a period of six hours. On the top are the wells that had been treated with spent culture supernatants, and then stained with CV. Even after periods as short as thirty minutes, the development of a biofilm is clearly apparent in the wells treated with spent culture supernatant. In addition we assayed for biofilm promoting activity after the following treatments: (1) No factor present; (2) Factor present; (3) 1 hr 50° C.; (4) pronase; (5) protease XIII; (6) 30 min autoclaving. The results are shown in FIG. 17.

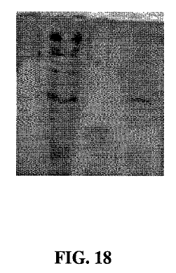

We tested certain physical properties of this biofilm promoting activity and found it to be proteinaceous in nature due to its sensitivity to treatments with a number of proteases. Starting from spent culture supernatants we purified the activity using standard protein purification protocols. In FIG. 18, this activity is shown as a single protein band in a silver stained SDS polyacrylamide gel. Surprisingly, all species that were tested responded to this factor, including Pseudomonas aeruginosa, Escherichia coli, Burkholderia cepacia, Agrobacterium tumefaciens, Staphylococcus aureus, and Staphyloccocus epidermidis.

Similar activities have been found in culture supernatants from a number of bacteria in addition to R. etli. Thus, it appears that several bacterial species make, upon entry into stationary phase, soluble extracellular factors that greatly stimulate bacterial aggregations and attachment to surfaces that leads to biofilm development.

Other Embodiments

Although the present invention has been described with reference to preferred embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention described herein. Such equivalents are intended to be encompassed in the scope of the present invention.

All publications, patents, and patent applications mentioned in this specification are herein incorporated by reference.

What is claimed is:

1. A compound comprising a chain of at least three rhamnose moieties linked to a lipid moiety and derivatives thereof.

2. The compound of claim 1, wherein said chain comprises seven rhamnose moieties.

3. The compound of claim 2, wherein said lipid moiety comprises a complex lipid containing an ester linkage.

4. A method for preventing a bacterium from participating in formation of a biofilm, said method comprising exposing said bacterium to a biological surfactant.

5. The method of claim 4, wherein said biological surfactant is a rhamnolipid compound.

6. The method of claim 5, wherein said rhamnolipid compound comprises a chain having at least three rhamnose moieties linked to a lipid moiety.

7. The method of claim 6, wherein said rhamnolipid compound comprises a chain of seven rhamnose moieties linked to a lipid moiety.

8. The method of claim 7, wherein said bacterium is selected from the group consisting of Pseudomonas fluorescens, Pseudomonas aeruginosa, Pseudomonas acidovorans, Pseudomonas alcaligenes, Pseudomonas putida, Pseudomonas syringae, Pseudomonas aureofaciens, Pseudomonas fragi, Fusobacterium nucleatum, Treponema denticola, Porphyromonas gingivalis, Moraxella catarrhalis, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudo tuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Pasteurella multocida, Pasteurella haemolytica, Helicobacter pylori, Campylobacter fetus, Campylobacter jejuni, Campylobacter coli, Borrelia burgdorferi, Vibrio cholerae, Vibrio paramaemolyticus, Legionella pneumophila, Listeria monocytogenes, Neisseria gonorrhoeae, Neisseria meningitidis, Gardnerella vaginalis, Bacteroides spp., Clostridium difficile, Mycobacterium tuberculosis, Mycobacterium avium, Mycobacterium intracellulare, Mycrobacterium leprae, Corynebacterium diphtheriae, Corynebacterium ulcerans, Streptococcus spp., Enterococcus spp., Desulfvibrio spp., Actinomyces spp., Erwinia spp., Xanthomonas spp., Xylella spp., Clavibacter spp., Desulfomonas spp., Desulfovibrio spp., Desulfococcus spp., Desulfobacter spp., Desulfobulbus spp., Desulfosarcina spp., Deslfuromonas spp., Bacillus spp., Streptomyces spp., Clostridium spp., Rhodococcus spp., Thermatoga spp., Sphingomonas spp., Zymomonas spp., Micrococcus spp., Azotobacter spp., Norcardia spp., Brevibacterium spp., Alcaligenes spp., Microbispora spp., Micromonospora spp., Methylobacterium organophilum, Pseudomonas reptilivora, Pseudomonas carragienovora, Pseudomonas dentificans, Corynebacterium spp., Propionibacterium spp., Xanothomonas spp., Methylobacterium spp., Chromobacterium spp., Saccharopolyspora spp., Actinobacillus spp., Alteromonas spp., Aeronomonas spp., Agrobacterium tumefaciens, Staphylococcus aureus, Staphylococcus epidennidis, Staphylococcus hominis, Staphylococcus haemolyticus, Staphylococcus warneri, Staphylococcus cohnii, Staphylococcus saprophyticus, Staphylococcus capitis, Staphylococcus lugdunensis, Staphylococcus intemedius, Staphylococcus hyicus, Staphylococcus saccharolyticus and Rhizobium spp., and mutant derivatives thereof.

9. The method of claim 8, wherein said bacterium is Pseudomonas aeruginosa.

10. The method of claim 8, wherein said bacterial organism is selected from Streptococcus spp.

11. The method of claim 8, wherein said bacterial organism is Staphylococcus aureus.

12. The method of claim 8, wherein said bacterial organism is Staphylococcus epidermis.

13. The method of claim 8, wherein said bacterial organism is a coagulase-negative Staphylococcus.

14. The method of claim 7, wherein said rhamnolipid compound is derived from supernatant of a closed culture system.

15. A biofilm modulating compound identified by the method comprising:
(a) obtaining supernatant from a closed culture system, said system comprising at least one type of a source microorganism;
(b) exposing a target organism to said supernatant or an extract thereof; and
(c) measuring the level of said activity,
wherein the compound is not an acyl homoserine lactone, a microbially-produced hydrolytic enzyme, a Lactobocillus biosurfactant, or an autoinducing compound.

16. The compound of claim 15, wherein said compound is purified from supernatant from a closed culture system.

17. The compound of claim 15, wherein said compound is synthesized.

18. A method of modulating a biofilm, said method comprising exposing a target organism to the compound of claim 15.

19. The method of claim 18, wherein said modulating comprises inhibition of biofilm formation.

20. The method of claim 18, wherein said modulating comprises disruption of a preexisting biofilm.

21. The method of claim 18, wherein said modulating comprises promoting biofilm formation.

22. The method of claim 18, wherein said modulating comprises decreasing the viability of cells within said biofilm.

23. The method of claim 18, wherein said modulating comprises potentiating the ability of an antimicrobial agent to decrease the viability of cells within said biofilm.

24. The method of claim 18, wherein said biofilm comprises at least one target organism selected from the group consisting of archaea, bacteria, fungi, protozoa, and algae.

25. The method of claim 24, wherein said organism is a bacterial organism.

26. The method of claim 25, wherein said bacterial organism is selected from the group consisting of Pseudomonas fluorescens, Pseudomonas aeruginosa, Pseudomonas acidovorans, Pseudomonas alcaligenes, Pseudomonas putida, Pseudomonas syringae, Pseudomonas aureofaciens, Pseudomonas fragi, Fusobacterium nucleatum, Treponema denticola, Porphyromonas gingivalis, Moraxella catarrhalis, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudo tuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Pasteurella multocida, Pasteurella haemolytica, Helicobacter pylori, Campylobacter fetus, Campylobacter jejuni, Campylobacter coli, Borrelia burgdorferi, Vibrio cholerae, Vibrio paramaemolyticus, Legionella pneumophila, Listeria monocytogenes, Neisseria gonorrhoeae, Neisseria meningitidis, Gardnerella vaginalis, Bacteroides spp., Clostridium difficile, Mycobacterium tuberculosis, Mycobacterium avium, Mycobacterium intracellulare, Mycrobacterium leprae, Corynebacterium diphtheriae, Corynebacterium ulcerans, Streptococcus spp., Enterococcus spp., Desulfvibrio spp., Actinomyces spp., Erwinia spp., Xanthomonas spp., Xylella spp., Clavibacter spp., Desulfomonas spp., Desulfovibrio spp., Desulfococcus spp., Desulfobacter spp., Desulfobulbus spp., Desulfosarcina spp., Deslfuromonas spp., Bacillus spp., Streptomyces spp., Clostridium spp., Rhodococcus spp., Thermatoga spp., Sphingomonas spp., Zymomonas spp., Micrococcus spp., Azotobacter spp., Norcardia spp., Brevibacterium spp., Alcaligenes spp., Microbispora spp., Micromonospora spp., Methylobacterium organophilum, Pseudomonas reptilivora, Pseudomonas carragienovora, Pseudomonas dentificans, Corynebacterium spp., Propionibacterium spp., Xanothomonas spp., Methylobacterium spp., Chromobacterium spp., Saccharopolyspora spp., Actinobacillus spp., Alteromonas spp., Aeronomonas spp., Agrobacterium tumefaciens, Staphylococcus aureus, Staphylococcus epidennidis, Staphylococcus hominis, Staphylococcus haemolyticus, Staphylococcus warneri, Staphylococcus cohnii, Staphylococcus saprophyticus, Staphylococcus capitis, Staphylococcus lugdunensis, Staphylococcus intermedius, Staphylococcus hyicus, Staphylococcus saccharolyticus and Rhizobium spp., and mutant derivatives thereof.

27. The method of claim 26, wherein said bacterial organism is Pseudomonas aeruginosa.

28. The method of claim 26, wherein said bacterial organism is selected from Streptococcus spp.

29. The method of claim 26, wherein said bacterial organism is Staphylococcus aureus.

30. The method of claim 26, wherein said bacterial organism is Staphylococcus epidermis.

31. The method of claim 25, wherein said bacterial organism is a coagulase-negative Staphylococcus.

32. The method of claim 24, wherein said target organism is a fungal organism.

33. The method of claim 32, wherein said fungal organism is selected from the group consisting of Absidia spp., Actinomadura madurae, Actinomyces spp., Allescheria boydii, Alternaria spp., Anthopsis deltoidea, Aphanomyces spp., Apophysomyces eleqans, Armillaria spp., Arnium leoporinum, Aspergillus spp., Aureobasidium pullulans, Basidiobolus ranarum, Bipolaris spp., Blastomyces dermatitidis, Botrytis spp., Candida spp., Centrospora spp., Cephalosporium spp., Ceratocystis spp., Chaetoconidium spp., Chaetomium spp., Cladosporium spp., Coccidioides immitis, Colletotrichum spp, Conidiobolus spp., Corynebacterium tenuis, Cryptoporiopsis spp., Cylindrocladium spp., Cryptococcus spp., Cunninghamella bertholletiae, Curvularia spp., Dactylaria spp., Diplodia spp., Epidermophyton spp., Epidermophyton floccosum, Exserophilum spp., Exophiala spp., Fonsecaea spp., Fulvia spp., Fusarium spp., Geotrichum spp., Guignardia spp., Helminthosporium spp., Histoplasma spp., Lecythophora spp., Macrophomina spp., Madurella spp., Magnaporthe spp., Malassezia furfur, Microsporum spp., Monilinia spp., Mucor spp., Mycocentrospora acerina, Nectria spp., Nocardia spp., Oospora spp., Ophiobolus spp., Paecilomyces spp., Paracoccidioides brasiliensis, Penicillium spp., Phaeosclera dematioides, Phaeoannellomyces spp., Phialemonium obovatum, Phialophora spp., Phlyctaena spp., Phoma spp., Phomopsis spp., Phymatotrichum spp., Phytophthora spp., Pythium spp., Piedraia hortai, Pneumocystis carinii, Puccinia spp., Pythium insidiosum, Rhinocladiella aquaspersa, Rhizomucor pusillus, Rhizoctonia spp., Rhizopus spp., Saccharomyces spp., Saksenaea vasifornis, Sarcinomyces phaeomuriformis, Scerotium spp., Sclerotinia spp., Sphaerotheca spp., Sporothrix schenckii, Syncephalastrum racemosum, Taeniolella boppii, Taphrina spp., Thielaviopsis spp., Torulopsosis spp., Trichophyton spp., Trichosporon spp., Ulocladium chartarum, Ustilago spp., Venturia spp., Verticillium spp., Wangiella dernatitidis, Whetxelinia spp., Xylohypha spp., and their synonyms.

34. The method of claim 33, wherein said fungal organism is selected from Candida spp.

35. A method of potentiating the ability of an antimicrobial agent to decrease the viability of a microorganism within a biofilm, said method comprising exposing said microorganism to the supernatant of a closed culture system or an extract thereof.

36. The method of claim 35, wherein said biological surfactant is a rhamnolipid compound.

37. The method of claim 36, wherein said rhamnolipid compound comprises a chain having at least three rhamnose moieties linked to a lipid moiety.

38. The method of claim 37, wherein said rhamnolipid compound comprises a chain of seven rhamnose moieties linked to a lipid moiety.

39. The method of claim 38, wherein said microorganism is a bacterium selected from the group consisting of Pseudomonas fluorescens, Pseudomonas aeruginosa, Pseudomonas acidovorans, Pseudomonas alcaligenes, Pseudomonas putida, Pseudomonas syringae, Pseudomonas aureofaciens, Pseudomonas fragi, Fusobactelium nucleatum, Treponema denticola, Porphyromonas gingivalis, Moraxella catarrhalis, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudo tuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Pasteurella multocida, Pasteurella haemolytica, Helicobacter pylori, Campylobacter fetus, Campylobacter jejuni, Campylobacter coli, Borrelia burgdorferi, Vibrio cholerae, Vibrio paramaemolyticus, Legionella pneumophila, Listeria monocytogenes, Neisseria gonorrhoeae, Neisseria meningitidis, Gardnerella vaginalis, Bacteroides spp., Clostridium difficile, Mycobacterium tuberculosis, Mycobacterium avium, Mycobacterium intracellulare, Mycrobacterium leprae, Corynebacterium diphtheriae, Corynebacterium ulcerans, Streptococcus spp., Enterococcus spp., Desulfvibrio spp., Actinomyces spp., Erwinia spp., Xanthomonas spp., Xylella spp., Clavibacter spp., Desulfomonas spp., Desulfovibrio spp., Desulfococcus spp., Desulfobacter spp., Desulfobulbus spp., Bacillus spp., Streptomyces spp., Clostridium spp., Rhodococcus spp., Thermatoga spp., Sphingomonas spp., Zymomonas spp., Micrococcus spp., Azotobacter spp., Norcardia spp., Brevibacterium spp., Alcaligenes spp., Microbispora spp., Micromonospora spp., Methylobacterium organophilum, Pseudomonas reptilivora, Pseudomonas carragienovora, Pseudomonas dentificans, Corynebacterium spp., Propionibacterium spp., Xanothomonas spp., Methylobacterium spp., Chromobacterium spp., Saccharopolyspora spp., Actinobacillus spp., Alteromonas spp., Aeronomonas spp., Desulfosarcina spp., Deslfuromonas spp., Agrobacterium tumefaciens, Staphylococcus aureus, Staphylococcus epidermidis, Staphylococcus hominis, Staphylococcus haemolyticus, Staphylococcus warneri, Staphylococcus cohnii, Staphylococcus saprophyticus, Staphylococcus capitis, Staphylococcus lugdunensis, Staphlyococcus intermedius, Staphylococcus hyicus, Staphylococcus saccharolyticus and Rhizobium spp., and mutant derivatives thereof.

40. The method of claim 39, wherein said bacterium is Pseudomonas aeruginosa.

41. The method of claim 39, wherein said bacterial organism is selected from Streptococcus spp.

42. The method of claim 39, wherein said bacterial organism is Staphylococcus aureus.

43. The method of claim 39, wherein said bacterial organism is Staphylococcus epidermis.

44. The method of claim 39, wherein said bacterial organism is a coagulase-negative Staphylococcus.

45. The method of claim 35, wherein said rhamnolipid compound is derived from supernatant of a closed culture system.
